(19) United States
(12) Patent Application Publication
(10) Pub. No.: US 2026/0027026 A1
(43) Pub. Date: Jan. 29, 2026

(54) COLOR DEPOSITION ENHANCEMENT OF NON-OXIDATIVE HAIR DYE

(71) Applicant: Henkel AG & Co. KGaA, Duesseldorf (DE)

(72) Inventor: Angeline B. Louis, West Hartford, CT (US)

(21) Appl. No.: 19/267,753

(22) Filed: Jul. 14, 2025

Related U.S. Application Data
(60) Provisional application No. 63/675,286, filed on Jul. 25, 2024.

(51) Int. Cl. A61K 8/34; A61K 8/86; A61Q 5/10

(52) U.S. Cl. CPC A61K 8/345; A61K 8/86; A61Q 5/10

(57) ABSTRACT

A dye composition for non-oxidative hair coloration, including a non-oxidative hair dye; an alkanediol; an ether; and water, wherein the weight ratio of the alkanediol to the ether is 3:1 to 1:3, and wherein the weight ratio of the hair dye to the total weight of the alkanediol and the ether is 1:7 to 2:1.

### CROSS-REFERENCE TO RELATED APPLICATION

This application claims priority to and the benefit of U.S. Provisional Patent Application No. 63/675,286, filed Jul. 25, 2024. The related application is incorporated herein by reference in its entirety.

### BACKGROUND

The present disclosure relates to hair dye compositions for the semi-permanent dyeing of keratin fibers, in particular human hair, methods of manufacture, and methods of use thereof.

Oxidative hair dyes are usually used for permanent hair coloration and provide intense color and good color fastness. Oxidative hair dyes usually include oxidation dye precursors and oxidizing agents leading to long-lasting color results and some degree of hair damage. On the other hand, non-oxidative hair dyes typically provide temporary color deposition on hair with minimal hair damage. The semi-permanent hair coloration from non-oxidative hair dyes generally only lasts between 3 to 8 washes after application due to the inability of the non-oxidative hair dye to penetrate the hair fiber.

Therefore, a need remains for a non-oxidative hair dye suitable for depositing more dye on the hair fiber to prolong the hair coloration results.

### SUMMARY

Disclosed, in various embodiments, is a dye composition for non-oxidative hair coloration, including a non-oxidative hair dye; an alkanediol; an ether; and water. The weight ratio of the alkanediol to the ether is 3:1 to 1:3 and the weight ratio of the hair dye to the total weight of the alkanediol and the ether is 1:7 to 2:1.

In another embodiment, a method of making a dye composition includes mixing a non-oxidative hair dye, an alkanediol, an ether, and water to provide the dye composition. The weight ratio of the alkanediol to the ether is 3:1 to 1:3 and the weight ratio of the hair dye to the total weight of the alkanediol and the ether is 1:7 to 2:1.

In another embodiment, a method of using a dye composition includes providing the dye composition; applying the dye composition to a keratin fiber to provide a dye-deposited keratin fiber; processing the dye-deposited keratin fiber to provide a processed keratin fiber; and rinsing the processed keratin fiber to provide the dyed keratin fiber. The dye composition comprises a non-oxidative hair dye, an alkanediol, an ether, and water, wherein the weight ratio of the alkanediol to the ether is 3:1 to 1:3 and the weight ratio of the hair dye to the total weight of the alkanediol and the ether is 1:7 to 2:1.

These and other features and characteristics are more particularly described below.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Disclosed herein is a dye composition for non-oxidative hair coloration. The dye composition comprises a non-oxidative hair dye with a mixture of solubilizers in an aqueous solution capable of depositing more dye on a hair fiber to prolong the colorfastness than a comparable dye composition without the selected mixture of solubilizers. The solubilizer mixture of the dye composition comprises an alkanediol solubilizer and ether solubilizer. The mixture of the alkanediol and the ether unexpectedly provides more dye deposition than dye deposition in the presence of the alkanediol or the ether alone. In addition, the disclosed dye composition can provide more dye deposition at a lower overall dye concentration than a dye composition with a higher dye concentration without the selected mixture of solubilizers. The dye composition can be applied directly to hair fiber at full strength or diluted into a shampoo or conditioner chassis carrier. Dyed hair fiber colored with the disclosed dye composition can exhibit prolonged colorfastness. For example, the dyed hair fiber can exhibit a CIELAB color space L* value of greater than 10 after up to 25 hair washes.

“Hair fiber” or “hair”, as used herein, refers to keratin fibers, such as furs, wool, feathers, and human hair.

### Dye Composition

The disclosed dye composition for non-oxidative hair coloration comprises a non-oxidative hair dye, an alkanediol, an ether, and water. The weight ratio of the alkanediol to the ether can be 5:1 to 1:5, 4:1 to 1:4, 3:1 to 1:3, 1:1 to 1:3, 3:1 to 1:1, 2:1 to 1:2, 1:2 to 1:3, or 3:1 to 2:1 (alkanediol:ether). The dye composition can be diluted into a chassis. The chassis may be a liquid, cream, or gel that includes water to serve as a carrier or diluent for the dye composition. In some embodiments, the chassis may be a shampoo, a conditioner, a masque, or a combination thereof. “Shampoo”, as used herein, refers to a cleaning liquid, cream, or gel that comprises a soap or detergent to wash hair. “Conditioner”, as used herein, refers to a liquid, cream, or gel that comprises a silicone, a fatty alcohol, a quaternary ammonium compound, or the like that serves to condition hair by reducing the overall negative charge on hair fibers after washing. “Masque”, as used herein, refers to a deep conditioning treatment that is left on the hair fiber for an extended period of time (e.g., 10 minutes). Typically, masques are thicker and more concentrated than conditioners. The dye composition may be combined with the chassis in a weight ratio of 99:1 to 1:99, 20:1 to 1:20, or 3:7 (dye composition:chassis).

The dye composition may be free of oxidative dyes, oxidative dye precursors, oxidizing agents, or the like. In an aspect, the dye composition has less than 100 ppm, less than 50 ppm, less than 10 ppm, or less than 1 ppm of an oxidative dye, an oxidative dye precursor, an oxidizing agent, or a combination thereof. “Oxidative dye”, as used herein refers to a hair dye composition including an oxidizing agent. “Oxidative dye precursor”, as used herein refers to a dye compound that reacts in the presence of an oxidizing agent to dye hair, such as para- or meta-substituted aminophenyl compounds and phenol compounds. Examples of an oxidizing agent include peroxides (e.g., hydrogen peroxide, calcium peroxide). In an aspect, the dye composition may comprise less than 1 ppm of a peroxide. The dye composition may comprise less than 1 ppm of hydrogen peroxide.

### Non-Oxidative Hair Dye

As used herein, “non-oxidative hair dye” refers to a direct dye that typically provides temporary hair color. Non-oxidative hair dyes generally do not require an oxidizing agent to dye hair and they dye hair directly by depositing color on the hair fiber. The disclosed non-oxidative hair dye may comprise an acidic (anionic), a basic (cationic), a non-ionic hair dye, or a combination thereof. The non-oxidative hair dye may have a low molecular weight. The molecular weight of the non-oxidative hair day may be 100 grams per mole (g/mol) to 1,000 g/mol, or less than 600 g/mol. The weight ratio of the non-oxidative hair dye to the total weight of the alkanediol and ether may be 1:7 to 2:1, or 1:4 to 1:2 [hair dye:(alkanediol and ether)]. The non-oxidative hair dye may comprise 0.001 weight percent (wt %) to 15 wt %, 0.01 wt % to 10 wt %, 0.1 wt % to 8 wt %, or 0.3 wt % to 7 wt % of the total weight of the dye composition. Each of the acidic, the basic, and the non-ionic hair dye may comprise 0.001 weight percent (wt %) to 15 wt %, 0.01 wt % to 10 wt %, 0.1 wt % to 8 wt %, or 0.3 wt % to 7 wt % of the total weight of the dye composition. When more than one of the acidic, the basic, or the non-ionic hair dye comprise the non-oxidative hair dye, the total combined weight percent of the hair dye of the more than one of the acidic, the basic, or the non-ionic hair dye may together comprise 0.001 weight percent (wt %) to 15 wt %, 0.01 wt % to 10 wt %, 0.1 wt % to 8 wt %, or 0.3 wt % to 7 wt % of the total weight of the dye composition.

The non-oxidative hair dye can be a nitrophenylenediamine, a nitroaminophenol, an azo dye, an anthraquinone, a triarylmethane dye, an indophenol, or their physiologically compatible salts.

The overall structure of the acidic dye compound carries an anionic charge. The anionic acidic dyes can comprise a dye compound with acidic groups, such as carboxylic acid groups, sulfonic acid groups, or a combination thereof.

Suitable acidic dyes include Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (CI 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; no sodium salt; Brown No. 201; RESORCIN BROWN; ACID ORANGE 24; Japan Brown 201; D & C Brown No. 1), Acid Red 14 (CI14720), Acid Red 18 (E124, Red 18; CI 16255), Acid Red 27 (E 123, CI 16185, C-Rot 46, Echtrot D, FD&C Red Nr. 2, Food Red 9, Naphtholrot S), Acid Red 33 (Red 33, Fuchsia Red, D&C Red 33, CI 17200), Acid Red 35 (CI CI18065), Acid Red 51 (CI 45430, Pyrosin B, Tetraiodofluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (D&C Red; Red 104; AKA231; RED 28; SUREDYE; 11969 Red; PHLOXINE; CI 45405; CI 45410; EOSINE B); Acid Red 95 (CI 45425, Erythrosine, Simacid Erythrosine Y), Acid Red 184 (CI 15685), Acid Red 195; Pigment Red 57:1 (E180; D & CRED7; CI 15850; Rubine 4BN; CI 15850:1; PIGMENT RED 57; LithoIrubine BK; LITHOLRUBINE RB; LITHOLRUBINE BCA; Lithol Rubine B); Acid Violet 43 (Jarocol Violet 43, Ext. D&C Violet no 2, CI 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Bluc 104 (CI 42735), Acid Blue 9 (E 133, Patent Blue AE, Amido Blue AE, Erioglaucine A, CI 42090, CI Food Bluc 2), Acid Bluc 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (CI 42100), Acid Green 22 (CI 42170), Acid Green 25 (CI 61570, Japan Green 201, D&C Green No. 5), Acid Green 50 (Brilliant Acid Green BS, CI 44090, Acid Brilliant Green BS, E 142), Acid Black 1 (Black no 401, Naphthalene Black 10B, Amido Black 10B, CI 20470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Brown 1, bromophenol blue, tetrabromophenol blue, or a combination thereof.

The overall structure of the basic dye compound carries a cationic charge. The cationic dyes can comprise a dye compound with basic groups, such as tertiary amine groups, quaternary amine groups, or a combination thereof.

Suitable basic (cationic) dyes include Basic Bluc 6 (CI-No. 51,175), Basic Bluc 7 (CI-No. 42,595) Basic Bluc 9 (CI-No. 52,015), Basic Bluc 26 (CI-No. 44,045), Basic Bluc 41 (CI-No. 11,154), Basic Bluc 99 (CI-No. 56,059), HC Blue 15, HC Blue 16 (Blue Quat Bromide), Cationic Blue 347, Basic Brown 4 (CI-No. 21,010), Basic Brown 16 (CI-No. 12,250), Basic Brown 17 (CI-No. 12,251), Natural Brown 7 (CI-No. 75,500), Basic Green 1 (CI-No. 42,040), Basic Red 2 (CI-No. 50,240), Basic Red 22 (CI-No. 11,055), Basic Red 51, Basic Red 76 (CI-No. 12,245), Basic Violet 1 (CI-No. 42,535), Basic Violet 2, Basic Violet 3 (CI-No. 42,555), Basic Violet 10 (CI-No. 45,170), Basic Violet 14 (CI-No. 42,510), Basic Yellow 57 (CI-No. 12,719), Basic Yellow 87, Basic Orange 31, or a combination thereof.

The overall structure of a non-ionic dye compound carries a neutral charge. Suitable non-ionic dyes include HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, HC Blue 15, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)-amino-2-nitrobenzene, 3-nitro-4-(2-hydroxyethyl) aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and its salts, N,N′-bis(2-hydroxyethyl)-2-nitro-p-phenylenediamine, 2-amino-6-chloro-4-nitrophenol, 4-ethylamino-3-nitrobenzoic acid, 2-chloro-6-ethylamino-4-nitrophenol, or a combination thereof.

### Alkanediol

Due to their hydrophilic head groups and lipophilic alkyl chains, alkanediols have amphiphilic properties. These amphiphilic properties can enable the alkanediol to couple with organic solvents, such as ethers. Furthermore, alkanediols can act as penetration enhancers for the non-oxidative dye into the hair fiber, as alkanediols can serve to increase the solubility of the non-oxidative dye.

The weight ratio of the alkanediol to the ether may be 5:1 to 1:5, 4:1 to 1:4, 3:1 to 1:3, 1:1 to 1:3, 3:1 to 1:1, 2:1 to 1:2, 1:2 to 1:3, or 3:1 to 2:1. The alkanediol may comprise 0.001 weight percent (wt %) to 25 wt %, 0.01 wt % to 15 wt %, 0.1 wt % to 10 wt %, or 0.3 wt % to 7 wt % of the total weight of the dye composition.

The alkanediol may be a C₂ to C₉ alkanediol or a polyethylene glycol with 3 to 20 ethylene oxide units. Suitable alkanediols include 1,2-ethanediol, 1,2-propanediol, 1,3-propanediol, 1,2-butanediol, 1,3-butanediol, 1,4-butanediol, 1,2-pentanediol, 1,3-pentanediol, 1,5-pentanediol, 1,2-hexanediol, 1,3-hexanediol, 1,6-hexanediol, 1,2-heptanediol, 1,3-heptandiol, 1,4-heptandiol, 1,7-hetanediol, 1,2-octanediol, 1,3-octanediol, 1,4-octanediol, 1,8-octanediol, dipropylene glycol, tripropylenglycol, cis-1,4-dimethylolcyclohexane, trans-1,4-dimethylolcyclohexane, PEG-3, PEG-4, PEG-6, PEG-7, PEG-8, PEG-9, PEG-10, PEG-12, PEG-14, PEG-16, PEG-18, PEG-20, or a combination thereof.

### Ether

Ether may serve as a solubilizer in the presence of the alkanediol. In an embodiment, the ether may comprise 2 to 40 ether functional groups. “Ether”, as used herein refers to the functional group R—O—R′, where R and R′ may be an alkyl group, an aryl group, a heteroaryl group, or a combination thereof. R and R′ may be the same or different. R and R′ may be independently selected from a C₂ to C₂₀ alkyl group, a C₂ to C₂₀ alkenyl group, a C₂ to C₂₀ alkynyl group, a C₂ to C₂₀ alkenylene group, a C₂ to C₂₀ alkynylene group, a C₆ to C₂₀ or aryl group, or a C₁ to C₃₀ heteroaryl group.

The C₂ to C₂₀ alkyl groups may include substituted or unsubstituted ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl groups, or a combination thereof. The C₂ to C₂₀ alkenyl groups may include any of the C₂ to C₂₀ alkyl groups with at least one carbon-carbon double bond. The C₂ to C₂₀ alkynyl groups may include any of the C₂ to C₂₀ alkyl groups with at least one carbon-carbon triple bond. The C₂ to C₂₀ alkenylene group may be a bivalent C₂ to C₂₀ alkenyl group, such as ethylene, propylene, butylene, or the like. The C₂ to C₂₀ alkynylene group may be a bivalent C₂ to C₂₀ alkynyl group, such as ethynylene, propynylene, butynylene, or the like.

The C₆ to C₂₀ aryl groups may include substituted or unsubstituted phenyl groups, naphthyl groups, anthracenyl, pyrenyl, pyrrolyl, or the like.

The C₁ to C₂₀ heteroaryl group may include substituted or unsubstituted pyridyl, furanyl, thienyl, indolyl, or the like.

The substituents on the C₂ to C₂₀ alkyl groups, the C₂ to C₂₀ alkenyl group, the C₂ to C₂₀ alkynyl group, the C₆ to C₂₀ or aryl group, or the C₁ to C₃₀ heteroaryl group may include hydrogen, deuterium, fluorine, chlorine, bromine, iodine, an ester, an amide, an alcohol, an ether, a hydroxyl, or the like.

The ether may be a polyethoxylated fatty alcohol with a degree of ethoxylation from 2 to 29, an addition product of ethylene oxide and propylene oxide with one or more fatty alcohol, polyethylene glycols, or the like. As used herein, an ethoxylated fatty alcohol is an addition product of ethylene oxide with a fatty alcohol, where the degree of ethoxylation indicates the molar amount of ethylene oxide. Fatty alcohols may include decanol, lauryl alcohol, isotridecyl alcohol, myristyl alcohol, cetyl alcohol, palmoleyl alcohol, stearyl alcohol, oleyl alcohol, elaidyl alcohol, petroselinyl alcohol, linolyl alcohol, linolenyl alcohol, elacostearyl alcohol, arachyl alcohol, gadoleyl alcohol, behenyl alcohol, erucyl alcohol, brassidyl alcohol, or a combination thereof. Suitable ethers may include ethoxydiglycol, polyoxyethylene-20 polyoxypropylene-5 cetyl ether (PPG-5-Ceteth-20), PPG-6 Deceth-9, PPG-1 Trideceth-6, PPG-6 Deceth-4, PPG-1-Laureth-9; PPG-12-Laneth-50, PPG-5-Ceteth-20, PPG-12-Laneth-65, PPG-6 C12-18 PARETH-11, PPG-4 Laureth-2, PPG-4 Laureth-5, PPG-8 Deceth-6, PPG-6 C12-18 Pareth-11, PPG-1-Laureth-9, PPG-5-Ceteth-20, PPG-5-Laureth-5, PPG-26-Buteth-26, PPG-20 Buteth-30, PPG-5 Buteth-7, PPG-12 Buteth-16, PPG-2 Buteth-3, PPG-3 Buteth-5, PPG-28 Buteth-35, PPG-33 Buteth-45, PPG-7 Buteth-10 and PPG-24-Buteth-27, or a combination thereof.

The ether may comprise 0.001 weight percent (wt %) to 25 wt %, 0.01 wt % to 15 wt %, 0.1 wt % to 10 wt %, or 0.3 wt % to 7 wt % of the total weight of the dye composition.

### Method of Making the Dye Composition

The dye composition may be prepared by mixing a non-oxidative hair dye, an alkanediol, an ether, and water to provide the dye composition. The alkanediol to the ether may be combined in a weight ratio of 3:1 to 1:3 or 2:1 to 1:3 or 1:1 to 1:3 or 1:1 to 3:1. The method may further include combining the dye composition with a chassis carrier in a weight ratio of 99:1 to 1:99, 20:1 to 1:20, or 3:7.

The following examples are merely illustrative of the invention disclosed herein and are not intended to limit the scope thereof.

### Method of Using the Dye Composition

A dye composition may be provided where the dye composition includes a non-oxidative hair dye, an alkanediol, an ether and water. The weight ratio of the alkanediol to the ether may be 3:1 to 1:3 or 2:1 to 1:3 or 1:1 to 1:3 or 1:1 to 3:1. The dye composition may be applied to a keratin fiber to provide a dye-deposited keratin fiber. The dye-deposited keratin fiber may be processed to provide a processed keratin fiber. The processed keratin fiber may be rinsed to provide the dyed keratin fiber. Prior to applying the dye composition to the keratin fiber may include diluting the dye composition into a chassis carrier at a weight ratio of 99:1 to 1:99 before applying the diluted dye composition to the keratin fiber to provide the dye-deposited keratin fiber. Processing of the keratin fiber may include a resting time period, where the dye composition is allowed to penetrate and deposit the dye onto the keratin fiber for a certain amount of time. The processing of the dye-deposited keratin fiber may be performed for 1 minute to 1 hour, or 5 minutes to 30 minutes. When the keratin fiber is a bleached, natural white keratin fiber, the dyed keratin fiber may comprise a color deposited on the dyed keratin fiber, wherein the color on the dyed keratin fiber has a L* value of greater than 10 after 25 washes as measured for the CIELAB color space with a visible light spectrophotometer. The CIELAB color space is a color space defined by the International Commission on Illumination (CEI). The L* value represents lightness from black to white on a scale of zero to 100 wherein the larger the number the “lighter” the color. Both a* and b* values represent chromaticity with no specific numeric limits. A negative a* value corresponds with green, a positive a* value corresponds with red, a negative b* value corresponds with blue and a positive b* value corresponds with yellow. The larger the b* value, the more yellow the color is, the smaller the b* value, the bluer the color is. Similarly, the larger the a* value, the more red the color is, the smaller the a* value, the greener the color is.

“Wash”, as used herein, refers to water treatment of the dyed keratin fiber with water at 30° C. to 60° C., specifically 35° C. to 40° C., and a cleaning agent such as a shampoo, a conditioner, or a combination thereof for 1 to 15 minutes.

The disclosed dye composition provides a non-oxidative hair dye suitable for depositing more dye on the hair fiber to prolong the hair coloration results. The dye composition comprises a selected ratio of solubilizers (alkanediol and ether) that unexpectedly provides more dye deposition than dye deposited in the presence of the alkanediol or the ether alone. Further benefits include increased dye deposition at a lower overall dye concentration for the disclosed composition than comparable dye compositions with a higher dye concentration made without the selected solubilizer mixture of the alkanediol and the ether. Furthermore, hair fiber dyed with the disclosed dye composition exhibits prolonged colorfastness.

### EXAMPLES

### Materials & Methods

The component materials used in the examples are listed in Table 1.

TABLE 1

| Component | Description |
|---|---|
| DI water | Deionized water |
| ARIANOR | A basic (cationic) hair dye. HC Blue No. 16. |
| Caribbean Blue | |
| Hexiol | Solubilizer. CAS 6920-22-5. 1,2-hexanediol. |
| ACQUASOLV | Solubilizer. Proprietary mixture includes PPG-5- |
| | Ceteth-20 (polyoxyethylene-20 polyoxypropylene-5 |
| | cetyl ether). |
| TRANSCUTOL | Solubilizer. CAS No. 111-90-0. |
| CG | Synonyms: Ethoxydiglycol, diethylene glycol |
| | monoethyl ether (DEGEE). |
| CETIOL HE | Solubilizer. Proprietary formulation includes PEG-7 |
| | Glyceryl Cocoate. |
| COVAFIX 123 | Solubilizer. Proprietary formulation including |
| | propylene glycol, benzyl alcohol, and quanternium-80. |

The full strength hair dye formulations used in the examples are listed in Tables 2 to 4.

TABLE 2

| | Formulation (wt % of each component) | | | | | | |
|---|---|---|---|---|---|---|---|
| Component | 1 | 2 | 3 | 4 | 5 | 6 | 7 |
| DI water | 93 | 90 | 75 | 75 | 75 | 75 | 75 |
| ARIANOR | 7 | 10 | 5 | 5 | 5 | 5 | 5 |
| Caribbean Blue | | | | | | | |
| Hexiol | | | 20 | | | | |
| ACQUASOLV | | | | 20 | | | |
| TRANSCUTOL CG | | | | | 20 | | |
| CETIOL HE | | | | | | 20 | |
| COVAFIX 123 | | | | | | | 20 |
| Total wt % | 100 | 100 | 100 | 100 | 100 | 100 | 100 |

TABLE 3

| | Formulation (wt % of each component) | | | | | | | | | |
|---|---|---|---|---|---|---|---|---|---|---|
| Component | 8 | 9 | 10 | 11 | 12 | 13 | 14 | 15 | 16 | 17 |
| DI water | 75 | 75 | 75 | 75 | 75 | 75 | 75 | 75 | 75 | 75 |
| ARIANOR | 5 | 5 | 5 | 5 | 5 | 5 | 5 | 5 | 5 | 5 |
| Caribbean Blue | | | | | | | | | | |
| Hexiol | 10 | 7 | 5 | 10 | 7 | 5 | 10 | 7 | 5 | 10 |
| ACQUASOLV | 10 | 14 | 15 | | | | | | | |
| TRANSCUTOL CG | | | | 10 | 14 | 15 | | | | |
| CETIOL HE | | | | | | | 10 | 14 | 15 | |
| COVAFIX 123 | | | | | | | | | | 10 |
| Total wt % | 100 | 100 | 100 | 100 | 100 | 100 | 100 | 100 | 100 | 100 |

TABLE 4

| | Formulation (wt % of each component) | | | | | | | | | |
|---|---|---|---|---|---|---|---|---|---|---|
| Component | 18 | 19 | 20 | 21 | 22 | 23 | 24 | 25 | 26 | 27 |
| DI water | 75 | 75 | 75 | 75 | 75 | 75 | 75 | 75 | 75 | 75 |
| ARIANOR | 5 | 5 | 5 | 5 | 5 | 5 | 5 | 5 | 5 | 5 |
| Caribbean Blue | | | | | | | | | | |
| Hexiol | 7 | 5 | 14 | 15 | 14 | 15 | 14 | 15 | 14 | 15 |
| ACQUASOLV | | | 7 | 5 | | | | | | |
| TRANSCUTOL CG | | | | | 7 | 5 | | | | |
| CETIOL HE | | | | | | | 7 | 5 | | |
| COVAFIX 123 | 14 | 15 | | | | | | | 7 | 5 |
| Total wt % | 100 | 100 | 100 | 100 | 100 | 100 | 100 | 100 | 100 | 100 |

### Chassis Carrier Formulation Preparation

To facilitate penetration of the dye onto the hair fiber, the hair dye formulation can be diluted into a cationic conditioner (i.e., chassis carrier). Where indicated, hair dye formulations were diluted into the chassis carrier to provide 30 wt % of the dye formulation in the chassis carrier, based on a total weight of the dye formulation and the chassis carrier (3:7 ratio of hair dye formulation to chassis carrier). The final concentration of the hair dye in the chassis carrier was 1.5 wt % based on a total weight of the dye formulation and the chassis carrier.

### Hair Dyeing Procedure

The hair dye formulation was applied to bleached, natural white hair to provide dye-deposited hair. The hair dye applied to the hair was at 100% full strength or 30 wt % in the chassis carrier. The dye-deposited hair was processed for three minutes, rinsed with water and air dried to provide a hair swatch sample for testing (dyed hair).

### Color Deposition Measurement

Six locations on each hair swatch sample were measured with a X-Rite Ci64 Spectrophotometer and the data points were averaged. The spectrophotometer is a visible light spectrophotometer with a spectral range of 400 nm to 700 nm. The spectrophotometer measured the color deposited on the hair using the CIELAB color space (L*a*b*) scale to quantify color properties.

### Experiment 1: Color Deposition Based on Dye Concentration

Both Formulation 1 and Formulation 2 were separately used without dilution. The hair dye formulations were applied to dye hair as described in the Hair Dying Procedure. Color measurements for the dyed samples are provided in Table 5.

TABLE 5

| Hair Dye | Average | Average | Average |
|---|---|---|---|
| Formulation | L* value | a* value | b* value |
| Formulation 1 | 29.63 | 8.08 | −35.06 |
| Formulation 2 | 31.27 | 6.22 | −34.54 |

Average L*, b*, and a* values for six data points

As shown in Table 5, color deposition did not increase with increased dye concentration. Rather, the more concentrated Formulation 2 deposited less hair dye (e.g., a b* value of −35.06 for Formulation 1 versus a b* value of −34.54 for Formulation 2).

### Experiment 2: Color Deposition Based on Solubilizers

Formulations 3 to 27 were diluted into the chassis carrier as described in Chassis Carrier Formulation Preparation prior to color deposition testing. The results of the color deposition testing are provided in Table 6. Each solubilizer was tested alone (Formulations 3 to 6) and mixtures of solubilizers in 1:1, 2:1, and 3:1 ratios were assessed.

TABLE 6

| Formulation | Solubilizer Description | Average b* Value |
|---|---|---|
| 1 | No solubilizer, no chassis carrier | −35.06 |
| 3 | Hexiol only | −37.06 |
| 4 | ACQUASOLV only | −36.52 |
| 5 | TRANSCUTOL CG only | −36.56 |
| 6 | CETIOL HE only | −34.90 |
| 7 | COVAFIX 123 only | −33.27 |
| 8 | ACQUASOLV:Hexiol (1:1) | −37.04 |
| 9 | ACQUASOLV:Hexiol (2:1) | −37.18 |
| 10 | ACQUASOLV:Hexiol (3:1) | −36.50 |
| 11 | TRANSCUTOL CG:Hexiol (1:1) | −36.94 |
| 12 | TRANSCUTOL CG:Hexiol (2:1) | −36.93 |
| 13 | TRANSCUTOL CG:Hexiol (3:1) | −36.72 |
| 14 | CETIOL HE:Hexiol (1:1) | −32.96 |
| 15 | CETIOL HE:Hexiol (2:1) | −34.47 |
| 16 | CETIOL HE:Hexiol (3:1) | −35.08 |
| 17 | COVAFIX 123:Hexiol (1:1) | −32.75 |
| 18 | COVAFIX 123:Hexiol (2:1) | −34.62 |
| 19 | COVAFIX 123:Hexiol (3:1) | −29.27 |
| 20 | Hexiol:ACQUASOLV (2:1) | −35.92 |
| 21 | Hexiol:ACQUASOLV (3:1) | −34.05 |
| 22 | Hexiol:TRANSCUTOL CG (2:1) | −35.87 |
| 23 | Hexiol:TRANSCUTOL CG (3:1) | −35.41 |
| 24 | Hexiol:CETIOL HE (2:1) | −34.59 |
| 25 | Hexiol:CETIOL HE (3:1) | −33.43 |
| 26 | Hexiol:COVAFIX 123 (2:1) | −34.85 |
| 27 | Hexiol:COVAFIX 123 (3:1) | −33.60 |

When each solubilizer was tested alone, Hexiol provided the greatest penetration (Formulation 3, b* value of −37.06). Out of all the formulations tested, the most penetration was measured for Formulation 9 with a mixture of ACQUASOLV and Hexiol in a 2:1 ratio. The solubilized dye formulations provided increased color deposition in comparison to the baseline 7 wt % dye formulation 1 versus the solubilized dye formulations with a dye concentration in the chassis carrier of only 1.5 wt %. The presence of an ether, (e.g., ACQUASOLV and TRANSCUTOL), in combination with an alkanediol (e.g., Hexiol) provided superior color deposition in comparison to solubilizers with an ester (e.g., CETIOL) and an ammonium salt (e.g., COVAFIX 123). Therefore, the comparable and superior color deposition demonstrates the improved performance of the solubilized dye formulations.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A dye composition for non-oxidative hair coloration, comprising:
a non-oxidative hair dye;
an alkanediol;
an ether; and
water,
wherein the weight ratio of the alkanediol to the ether is 3:1 to 1:3, and
wherein the weight ratio of the hair dye to the total weight of the alkanediol and the ether is 1:7 to 2:1.

2. The dye composition of claim 1, wherein the non-oxidative hair dye is a basic hair dye.

3. The dye composition of claim 1, wherein the non-oxidative hair dye has a molecular weight of 100 grams per mole to 1,000 grams per mole.

4. The dye composition of claim 1, wherein the non-oxidative hair dye is a basic hair dye and the basic hair dye is Basic Blue 6, Basic Blue 7, Basic Blue 9, Basic Blue 26, Basic Blue, Basic Blue 99, HC Blue 15, HC Blue 16, Cationic Blue 347, Basic Brown 4, Basic Brown 16, Basic Brown 17, Natural Brown 7, Basic Green 1, Basic Red 2, Basic Red 22, Basic Red 51, Basic Red 76, Basic Violet 1, Basic Violet 2, Basic Violet 3, Basic Violet 10, Basic Violet 14, Basic Yellow 57, Basic Yellow 87, Basic Orange 31, or a combination thereof.

5. The dye composition of claim 1, wherein the dye composition comprises less than 1 ppm of a peroxide.

6. The dye composition of claim 1, wherein the weight ratio of the hair dye to the total weight of the alkanediol and the ether is 1:4 to 1:2 and the weight ratio of the alkanediol to the ether is 1:1 to 1:3.

7. The dye composition of claim 1, wherein the alkanediol is a C₂ to C₉ alkanediol.

8. The dye composition of claim 1, wherein the alkanediol is 1,2-ethanediol, 1,2-propanediol, 1,3-propanediol, 1,2-butanediol, 1,3-butanediol, 1,4-butanediol, 1,2-pentanediol, 1,3-pentanediol, 1,5-pentanediol, 1,2-hexanediol, 1,3-hexanediol, 1,6-hexanediol, 1,2-heptanediol, 1,3-heptandiol, 1,4-heptandiol, 1,7-hetanediol, 1,2-octanediol, 1,3-octanediol, 1,4-octanediol, 1,8-octanediol, dipropylene glycol, tripropylenglycol, cis-1,4-dimethylolcyclohexane, trans-1,4-dimethylolcyclohexane, PEG-3, PEG-4, or a combination thereof.

9. The dye composition of claim 1, wherein the ether comprises 2 to 40 ether functional groups.

10. The dye composition of claim 1, wherein the ether is ethoxydiglycol, polyoxyethylene-20 polyoxypropylene-5 cetyl ether, PPG-6 Deceth-9, PPG-1 Trideceth-6, PPG-6 Deceth-4, PPG-1-Laureth-9; PPG-12-Laneth-50, PPG-5-Ceteth-20, PPG-12-Laneth-65, PPG-6 C12-18 PARETH-11, PPG-4 Laureth-2, PPG-4 Laureth-5, PPG-8 Deceth-6, PPG-6 C12-18 Pareth-11, PPG-1-Laureth-9, PPG-5-Ceteth-20, PPG-5-Laureth-5, PPG-26-Buteth-26, PPG-20 Buteth-30, PPG-5 Buteth-7, PPG-12 Buteth-16, PPG-2 Buteth-3, PPG-3 Buteth-5, PPG-28 Buteth-35, PPG-33 Buteth-45, PPG-7 Buteth-10 and PPG-24-Buteth-27, or a combination thereof.

11. The dye composition of claim 1, wherein the dye composition is diluted into a chassis carrier.

12. The dye composition of claim 1, wherein the dye composition is diluted into the chassis carrier at a ratio of 99:1 to 1:99.

13. The dye composition of claim 1, wherein the non-oxidative hair dye comprises 0.001 weight percent to 15 weight percent of the total weight of the dye composition, the alkanediol comprises 0.001 weight percent to 25 weight percent of the total weight of the dye composition, and the ether comprises 0.001 weight percent to 25 weight percent of the total weight of the dye composition.

14. A method of making a dye composition, comprising:
mixing a non-oxidative hair dye, an alkanediol, an ether, and water to provide the dye composition,
wherein the weight ratio of the alkanediol to the ether is 3:1 to 1:3 and the weight ratio of the hair dye to the total weight of the alkanediol and the ether is 1:7 to 2:1.

15. The method of claim 14, further comprising combining the dye composition with a chassis carrier in a ratio of 99:1 to 1:99.

16. A method of using a dye composition, comprising:
providing the dye composition, wherein the dye composition comprises a non-oxidative hair dye, an alkanediol, an ether, and water, wherein the weight ratio of the alkanediol to the ether is 3:1 to 1:3 and the weight ratio of the hair dye to the total weight of the alkanediol and the ether is 1:7 to 2:1;
applying the dye composition to a keratin fiber to provide a dye-deposited keratin fiber;
processing the dye-deposited keratin fiber to provide a processed keratin fiber; and
rinsing the processed keratin fiber to provide the dyed keratin fiber.

17. The method of claim 16, wherein the dye composition is diluted into the chassis carrier at a ratio of 99:1 to 1:99 to provide a diluted dye composition, before applying the diluted dye composition to the keratin fiber to provide the dye-deposited keratin fiber.

18. The method of claim 16, wherein the processing of the dye-deposited keratin fiber is performed for 1 minute to 1 hour.

19. The method of claim 16, wherein the keratin fiber is a bleached, natural white keratin fiber and the dyed keratin fiber comprises a color deposited on the dyed keratin fiber, wherein the color on the dyed keratin fiber has a L* value of greater than 10 after 25 washes as measured for the CIELAB color space with a visible light spectrophotometer.
